(19) United States
(12) Patent Application Publication
(10) Pub. No.: US 2012/0294924 A1
(43) Pub. Date: Nov. 22, 2012

(54) Peptide-Lipid Conjugates And Uses Thereof

(76) Inventors: THOMAS TICE, INDIAN SPRINGS, AL (US); TORSTEN WOEHR, MARBLEHEAD, MA (US)

(21) Appl. No.: 13/454,211

(22) Filed: Apr. 24, 2012

Related U.S. Application Data
(60) Provisional application No. 61/480,596, filed on Apr. 29, 2011.

(51) Int. Cl. C07K 14/435; C07K 7/06; A61K 9/00; A61K 9/14; A61P 31/04; A61K 38/16; A61K 38/10; A61K 38/08; A61K 38/02; C07K 7/08; A61K 9/127

(52) U.S. Cl. 424/422; 530/324; 530/325; 530/326; 530/328; 530/327; 530/330; 424/484; 424/499; 424/400; 424/450; 514/21.3; 514/21.4; 514/21.6; 514/21.8; 514/1.1; 514/2.4; 424/486

(57) ABSTRACT

Disclosed are peptide-lipid conjugates that bind lipopolysaccharide. Also disclosed are methods of making and using the peptide-lipid conjugates.

This application claims the benefit of U.S. Provisional Application No. 61/480,596, filed on Apr. 29, 2011, which is incorporated herein by reference in its entirety.

### BACKGROUND

Lipopolysaccharide (LPS) is an endotoxin present in the cell membrane of gram-negative bacteria. When bacteria invade an organism, LPS is released from the bacteria, which can lead to toxic effects. Even small amounts of LPS can lead to fatal septic shock syndrome, particularly if an inflammatory response resulting from the release of LPS is amplified or uncontrolled.

LPS can also contaminate biological samples and pharmaceutical formulations. LPS is frequently present as a contaminent in samples that have been prepared from bacteria, such as plasmid DNA, ovalbumin, and others. It is usually desirable to remove LPS from such samples and formulations prior to further use, especially if the samples or formulations are to be used in vivo.

While LPS detection and removal strategies have been developed, many of such strategies suffer from low detection sensitivity and irreproducibility. Additionally, therapies targeting LPS do not currently provide an optimal reduction in the incidence of LPS related complications that occur during a bacterial infection. As such, a need exists for improved LPS detection and removal strategies as well as new therapies targeting LPS.

### SUMMARY

The disclosed subject matter, in one aspect, relates to peptide-lipid conjugates that can bind lipopolysaccharide (LPS). Also disclosed are pharmaceutical compositions comprising the peptide-lipid conjugates, and methods of making and using the peptide-lipid conjugates.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 is a drawing illustrating exemplary peptide-lipid conjugates comprising Sushi 3 (SEQ ID NO: 3).

FIG. 2 is a schematic of an exemplary solid-phase method for preparing Sushi 3 (SEQ ID NO: 3).

FIGS. 3A, 3B, 4A, and 4B are schematics of exemplary methods for introducing a lipid onto an amino acid residue.

FIGS. 5, 6, and 7 are schematics of exemplary methods for introducing a lipid onto a peptide sequence such as Sushi 3 (SEQ ID NO: 3).

### DETAILED DESCRIPTION

The materials, compounds, compositions, and methods described herein may be understood more readily by reference to the following detailed description of specific aspects of the disclosed subject matter, the Figures, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Throughout this specification, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### GENERAL DEFINITIONS

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

### CHEMICAL DEFINITIONS

As used herein, the term “substituted” is contemplated to include all permissible substituents of organic compounds. In a broad aspect, the permissible substituents include organic and inorganic substitutients, including acyclic and cyclic, branched and unbranched, carbocyclic and heterocyclic, and aromatic and nonaromatic substituents. Illustrative substituents include, for example, those described below. The permissible substituents can be one or more and the same or different for appropriate organic compounds. For purposes of this disclosure, the heteroatoms, such as nitrogen and oxygen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Also, as used herein “substitution” or “substituted with” is meant to encompass configurations where one substituent is fused to another substituent. For example, an aryl group substituted with an aryl group (or vice versa) can mean that one aryl group is bonded to the second aryl group via a single sigma bond and also that the two aryl groups are fused, e.g., two carbons of one alkyl group are shared with two carbons of the other aryl group.

An “optionally substituted” residue or compound refers to a residue or compound that is either substituted or unsubstituted, wherein “substituted” is defined above.

“A¹,” “A²,” “A³,” and “A⁴” are used herein as generic symbols to represent various specific substituents. These symbols can be any substituent, not limited to those disclosed herein, and when they are defined to be certain substituents in one sentence it does not mean that, in another sentence, they cannot be defined as some other substituents.

The term “alkyl” as used herein is a branched or unbranched saturated hydrocarbon group of 1 to 40 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, octadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, azide, nitro, nitrile, or thiol, as described below. A “lower alkyl” is an alkyl group with up to six carbon atoms, e.g., methyl, ethyl, propyl, butyl, pentyl, and hexyl.

Throughout the specification “alkyl” is generally used to refer to both unsubstituted alkyl groups and substituted alkyl groups; however, substituted alkyl groups are also specifically referred to herein by identifying the specific substituents) on the alkyl group. For example, the term “alkyl halide” specifically refers to an alkyl group that is substituted with one or more halides, e.g., fluorine, chlorine, bromine, or iodine. When “alkyl” is used in one sentence and a specific term such as “alkyl halide” is used in another, it is not meant to imply that the term “alkyl” does not also refer to specific terms such as “alkyl halide” and the like.

This practice is also used for other groups described herein. That is, while a term such as “heteroaryl” refers to both unsubstituted and substituted heteroaryl moieties, the substituted moieties can, in addition, be specifically identified herein; for example, a particular substituted heteroaryl can be referred to as, e.g., an “alkyl heteroaryl.” Similarly, a substituted alkenyl can be, e.g., an “alkenyl halide,” and the like. Again, the practice of using a general term, such as “heteroaryl,” and a specific term, such as “alkyl heteroaryl,” is not meant to imply that the general term does not also include the specific term.

The term “alkoxy” as used herein is an alkyl or cycloalkyl group bonded through an ether linkage; that is, an “alkoxy” group can be defined as —OA¹ where A¹ is alkyl or cycloalkyl as defined above. A “lower alkoxy” is an alkoxy group with up to six carbon atoms, e.g., methoxy, ethoxy, propoxy, butoxy, pentoxy, and hexoxy.

The term “alkenyl” as used herein is a hydrocarbon group of from 2 to 60 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, azide, nitro, nitrile, or thiol.

The term “alkynyl” as used herein is a hydrocarbon group of 2 to 60 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, azide, nitro, nitrile, or thiol.

The term “aryl” as used herein is a group that contains any carbon-based aromatic group including, but not limited to, benzene, benzyl, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, sulfo-oxo, sulfonylamino, azide, nitro, nitrile, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, cyclooctyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, azide, nitrile, silyl, or thiol.

The term “cycloalkenyl” as used herein is a non-aromatic carbon-based ring composed of at least three carbon atoms and contains at least one double bound, e.g., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, sulfo-oxo, sulfonylamino, nitro, silyl, azide, nitrile, or thiol.

The term “cyclic group” is used herein to refer to either aryl groups (e.g., heteraryl, biaryl), non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula:

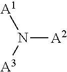

where A¹, A², and A³ can each be, independent of one another, hydrogen, an alkyl, halogenated alkyl, alkenyl, alkynyl, aryl, heteroaryl, cycloalkyl, cycloalkenyl, heterocycloalkyl, or heterocycloalkenyl group described above. Also, any of the A¹, A², and A³ substituents can be absent and any of the remaining substituents can be a multivalent group, i.e., form more than one bond with N.

The term “carboxylic acid” as used herein is represented by the formula —C(O)OH. The term “carboxylate” is a carboxylic acid that has been deprotonated, i.e., —C(O)O⁻. Protonation and deprotonation can be achieved by changes in pH. The terms “carboxylic acid” and “carboxylate” are understood to be interchangeable.

The term “ester” as used herein is represented by the formula —OC(O)A¹ or —C(O)OA¹, where A¹ can be a substituted or unsubstituted alkyl, cycloalkyl, alkenyl, cycloalkenyl, alkynyl, cycloalkynyl, aryl, or heteroaryl group as described herein.

The term “ether” as used herein is represented by the formula A¹OA², where A¹ and A² can be, independently, a substituted or unsubstituted alkyl, cycloalkyl, alkenyl, cycloalkenyl, alkynyl, cycloalkynyl, aryl, or heteroaryl group described herein.

The term “halide” as used herein refers to the halogens fluorine, chlorine, bromine, and iodine.

The term “hydroxyl” as used herein is represented by the formula —OH.

The term “ketone” as used herein is represented by the formula A¹C(O)A², where A¹ and A² can be, independently, a substituted or unsubstituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or a substituted or unsubstituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)²A¹, or —OS(O)₂OA¹, where A¹ can be hydrogen or a substituted or unsubstituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or a substituted or unsubstituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, a substituted or unsubstituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, a substituted or unsubstituted alkyl, cycloalkyl, alkenyl, cycloalkenyl, alkynyl, cycloalkynyl, aryl, or heteroaryl group as described herein.

The term “sulfonamide” as used herein is represented by the formula —S(O)₂NA¹-, where A¹ can be hydrogen, a substituted or unsubstituted alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” and “Rⁿ,” where n is some integer, as used herein can, independently, possess two or more of the groups listed above. For example, if R is a straight chain alkyl group, one of the hydrogen atoms of the alkyl group can optionally be substituted with a hydroxyl group (OH), an alkoxy group, halide, etc. Depending upon the groups that are selected, a first group can be incorporated within second group or, alternatively, the first group can be pendant (i.e., attached) or fused to the second group.

The terms “ortho,” “meta,” and “para” refer to 1,2-, 1,3-, and 1,4-disubstituted benzenes, respectively.

As used herein, the term “alcohol” refers to compounds having at least one hydroxyl group (—OH). The term “polyol” is used to specifically refer to alcohols having two (which can specifically be referred to as a “diol”) or more hydroxyl groups. Unless stated to the contrary the term “alcohol” is used herein to also refer to diols, triols, polyols and polymeric alcohols and polymeric polyols. Non-limiting examples of alcohols include methanol, ethanol, propanol, butanol, hexanol, octanol, decanol, dodecanol, oleyl alcohol, myristyl alcohol, cetyl alcohol, stearyl alcohol; short-chain alcohols (for example, C₁ to C₆ alcohols), medium-chain alcohols (for example, C₇ to C₁₂), long-chain alcohols (for example, C₁₃ to C₂₄ alcohols), and so on; saturated alcohols, unsaturated alcohols; benzyl alcohol, ethylene glycol, 1,3-propylene glycol, 1,2-propylene glycol, glycerol, and polymeric alcohols like modified polyvinyl alcohol, hydroxyl-containing PVP, and polyalkyleneoxy homo and copolymers, which can be alkoxy capped, for example, PEG, MPEG 600 and the like. Other examples of alcohols are disclosed elsewhere herein.

The disclosed lipids, when referred to as substituted, can comprise any of those substitutients discussed above, among others that do not undesirably alter the hydrophobicity of the lipid.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixtures.

### BIOLOGY DEFINITIONS

The terms “percent (%) sequence similarity,” “percent (%) sequence identity,” and the like, generally refer to the degree of identity or correspondence between different amino acid sequences of proteins or peptides that may or may not share a common evolutionary origin. Sequence identity can be determined using any of a number of publicly available sequence comparison algorithms, such as BLAST, FASTA, etc. To determine the percent identity between two amino acid sequences, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990, 87:2264, modified as in Karlin and Altschul, Proc. Natl. Acad. Sci. USA 90:5873-5877, 1993. Such an algorithm is incorporated into the NBLAST and XBLAST programs of Altschul et al., J. Mol. Biol. 215:403, 1990. BLAST protein searches can be performed with the XBLAST program, score=100, wordlength=12, to obtain amino acid sequences homologous to protein sequences of the invention. Another non-limiting example of a mathematical algorithm utilized for the comparison of sequences is the algorithm of Myers and Miller, CABIOS 4:11-17, 1988. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. In one embodiment, the percent identity between two amino acid sequences is determined using the algorithm of Needleman and Wunsch (J. Mol. Biol. 48:444-453, 1970), using either a Blossum 62 matrix or a PAM250 matrix, a gap weight of 16, 14, 12, 10, 8, 6, or 4, and a length weight of 1, 2, 3, 4, 5, or 6. Sequence similarity can also be determined by inspection.

As disclosed herein there are numerous variants of proteins and peptides (e.g., Sushi peptides) that are contemplated herein. In addition, to the known functional Sushi peptide variants there are derivatives of these proteins which also function in the disclosed methods and compositions. Protein variants and derivatives are well understood to those of skill in the art and can involve amino acid sequence modifications. For example, amino acid sequence modifications typically fall into one or more of three classes: substitutional, insertional, or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than from about 2 to about 6 residues are deleted at any one site within the protein molecule. These variants can ordinarily be prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Accordingly, recombinant technologies can be used for the production of the disclosed peptides. However, chemical synthesis is preferable for a relatively short peptide/protein such as Sushi-3 (SEQ ID NO: 3), for example. Amino acid substitutions are typically of single amino acid residues, but can occur at a number of different locations at once; insertions usually can be on the order of from about 1 to about 10 amino acid residues; and deletions can range from about 1 to about 30 residues. Deletions or insertions can be made in adjacent pairs, i.e., a deletion of 2 amino acid residues or insertion of 2 amino acid residues. Substitutions, deletions, insertions or any combination thereof may be combined to arrive at a final construct. Substitutional variants are those in which at least one amino acid residue has been removed and a different residue inserted in its place. Such substitutions generally are made in accordance with the following Table 1 and are referred to as conservative substitutions.

TABLE 1
Amino Acid Substitutions
Original Residue Exemplary Conservative
Substitutions, others are known in the art.

Ala [symbol] Ser
Arg [symbol] Lys; Gln
Asn [symbol] Gln; His
Asp [symbol] Glu
Cys [symbol] Ser
Gln [symbol] Asn or Lys
Glu [symbol] Asp
Gly [symbol] Pro
His [symbol] Asn or Gln
Ile [symbol] Leu or Val
Leu [symbol] Ile or Val
Lys [symbol] Arg or Gln
Met [symbol] Leu or Ile
Phe [symbol] Met, Leu, or Tyr
Ser [symbol] Thr
Thr [symbol] Ser
Trp [symbol] Tyr
Tyr [symbol] Trp or Phe
Val [symbol] Ile or Leu

Substantial changes in function can be made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another or one polar residue for another. The substitutions include combinations such as Gly, Ala; Val, Ile, Leu; Asp, Glu; Asn, Gln; Ser, Thr; Lys, Arg; and Phe, Tyr. Such conservatively substituted variations of each explicitly disclosed sequence are included within the polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g., Arg, can be accomplished, for example, by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the O-amino groups of lysine, arginine, and histidine side chains (Creighton, Proteins: Structure and Molecular Properties, W. H. Freeman & Co., San Francisco pp. 79-86 (1983), which is incorporated by reference herein for its material on post-translational derivatizations), acetylation of the N-terminal amine and, in some instances, amidation of the C-terminal carboxyl.

It is understood that one way to define the variants, derivatives, and analogs of the peptides and proteins disclosed herein is through defining the variants, derivatives, and analogs in terms of homology/identity to specific known sequences. For example, SEQ ID NO:3 sets forth the particular sequence of Sushi-3. Specifically disclosed are variants, derivatives, and analogs of these and other peptides and proteins herein disclosed which have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence similarity to the stated sequence. Those of skill in the art readily understand how to determine the sequence similarity of two proteins, as is disclosed more fully supra.

It is further understood that there are numerous amino acid and peptide analogs that can be incorporated into the disclosed compositions. For example, there are numerous D amino acids or amino acids which have a different functional substituent then the amino acids described above. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson, et al., Methods in Molec Biol 77:43-73, 1991, Zoller, Curr Opin Biotech 3:348-354, 1992; Ibba, Biotech & Gen Eng Rev 13:197-216, 1995, Cahill, et al., TIBS 14(10):400-403, 1989; Benner, TIB Tech 12:158-163, 1994; Ibba and Hennecke, Bio/technology 12:678-682, 1994, all of which are incorporated by reference herein for their material related to amino acid analogs).

It is further contemplated that molecules can be synthesized that resemble the peptides disclosed herein, but which are not connected via a natural peptide linkages. For example, peptide analogs can have linkages for amino acids or amino acid analogs that include —CH₂NH—, —CH₂S—, —CH₂—CH₂—, —CH═CH— (cis and trans), —COCH₂—, —CH(OH)CH₂—, and —CH₂SO— (These and others can be found in Spatola, in Chemistry and Biochemistry of Amino Acids, Peptides, and Proteins, B. Weinstein, eds., Marcel Dekker, New York, p. 267 (1983); Spatola, Vega Data (March 1983), Vol. 1, Issue 3, Peptide Backbone Modifications (general review); Morley, Trends Pharm Sci (1980) pp. 463-468; Hudson et al., Int J Pept Prot Res 14:177-185, 1979 (—CH₂NH—, —CH₂CH₂—); Spatola et al., Life Sci, 38:1243-1249, 1986 (—CH₂S—); Hann, J Chem Soc, Perkin Trans I, 307-314, 1982 (—CH═CH—, cis and trans); Almquist, et al., J Med Chem 23:1392-1398, 1980 (—COCH₂—); Jennings-White et al., Tetrahedron Lett 23:2533, 1982 (—COCH₂—); Szelke et al., European Appln, EP 45665 CA (1982) (—CH(OH)CH₂—); Holladay et al., Tetrahedron Lett 24:4401-4404, 1983 (—CH(OH)CH₂—); and Hruby Life Sci 31:189-199, 1982 (—CH₂S—); each of which is incorporated by reference herein for its material regarding peptide analogs, mimetics, and non-peptide linkages). Also, it is understood that peptide analogs can have more than one atom between the bond atoms, such as β-alanine, γ-aminobutyric acid, and the like.

Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others. For example, D-amino acids and β-amino acids can be used to generate more stable peptides, because these amino acids are not recognized by peptidases and such. Systematic substitution of one or more amino acids of a consensus sequence with a D- or β-amino acid of the same type (e.g., D-lysine in place of L-lysine or β-alanine in place of alanine) can be used to generate more stable peptides. Cysteine residues can be used to cyclize or attach two or more peptides together. This can be beneficial to constrain peptides into particular conformations (Rizo and Gierasch, Ann Rev Biochem 61:387, 1992).

Reference will now be made in detail to specific aspects of the disclosed materials, compounds, compositions, components, devices, articles, and methods, examples of which are illustrated in the following description and examples, and in the figures and their previous and following description.

### Materials and Methods

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Factor C protein in horseshoe crab is an enzyme that acts as a very sensitive biosensor responding to low level infection with Gram negative bacteria (SEQ ID NO:15) (see accession nos. AAB34362 and AAB34361). The N terminal region of Factor C, which comprises a number of repeating units of Sushi domains of about 60 amino acids each, is directly involved in the molecular recognitions process and binds to the negatively charged lipopolysaccharide (LPS) displayed on the bacteria cell surface. The LPS binding region is believed to be contained in one of the Sushi domains of Factor C. Sushi-3 (SEQ ID NO:3), for example, has been shown to bind to LPS (Li et al., Biochimica et Biophysica Acta 1768:411-418, 2007, which is incorporated by reference herein in its entirety).

Without wishing to be bound by theory, it is believed that the mechanism of action of the disclosed peptides includes an interaction of a positively charged N-terminal region with surface displayed LPS moieties of Gram negative bacteria. The initial binding by the peptide enables the peptide to perturb, possibly with its lipophilic C-terminus, the bacterial membrane and disrupt its fluidic integrity. Thus, the disclosed peptides can be useful in detecting, removing, and treating disorders associated with LPS.

### Peptides

Generally, the disclosed peptides include natural and synthetic peptide sequences from horse-shoe crab Factor C protein and variations thereof. The peptides can be identified and selected by their ability to bind to lipopolysaccharide (LPS). Methods for preparing factor C are disclosed in U.S. Pat. No. 5,716,834 (entitled “Cloned factor C cDNA of the Singapore horseshoe crab, Carcinoscorpius rotundicauda and purification of factor C proenzyme,” which is incorporated by reference herein for its teachings of factor C and methods for obtaining factor C). Methods for determining the binding efficacy of the peptides can be determined according to methods described in U.S. Pat. No. 6,719,973 to Ding et al., which is incorporated herein by this reference in its entirety for its teachings of Sushi peptides, methods of identifying, making, and obtaining them, and methods of using them.

The disclosed peptides include those with natural, unnatural, or non-amino acid residues. Synthetic peptides, for example, include those with modified amino acids or other moieties in place of an amino acid. The inclusion of unnatural or non-amino acids can be made to stabilize the peptide, block metabolization, or to create a conformational change in the peptide which would increase its effectiveness. Preferably, the amino acids of the peptides are in the L-orientation, although amino acids or peptides in the D-orientation can also be used, as can peptides in the reverse orientation.

The peptides can be from 3 to 100 amino acids in length, preferably 3 to 50, and more preferably 3 to 40 in length. Preferred peptides include those disclosed in U.S. Pat. No. 6,719,973 to Ding et al. Specific examples of the peptides are those that include the following sequences:

| (SEQ ID NO: 1) | |
|---|---|
| a. | (S1, Sushi-1) Gly-Phe-Lys-Leu-Lys-Gly-Met-Ala-Arg-Ile-Ser-Cys-Leu-Pro-Asn- |
| | Gly-Gln-Trp-Ser-Asn-Phe-Pro-Pro-Lys-Cys-Ile-Arg-Glu-Cys-Ala-Met-Val-Ser-Ser |
| (SEQ ID NO: 2) | |
| b. | (SΔ1) Gly-Phe-Lys-Leu-Lys-Gly-Lys-Ala-Lys-Ile-Ser-Cys-Leu-Pro-Asn-Gly-Gln- |
| | Trp-Ser-Asn-Phe-Pro-Pro-Lys-Cys-Ile-Arg-Glu-Cys-Ala-Met-Val-Ser-Ser |
| (SEQ ID NO: 3) | |
| c. | (S3, Sushi-3) His-Ala-Glu-His-Lys-Val-Lys-Ile-Gly-Val-Glu-Gln-Lys-Tyr-Gly-Gln- |
| | Phe-Pro-Gln-Gly-Thr-Glu-Val-Thr-Tyr-Thr-Cys-Ser-Gly-Asn-Tyr-Phe-Leu-Met |
| (SEQ ID NO: 4) | |
| d. | (SΔ3) His-Ala-Glu-His-Lys-Val-Lys-Ile-Lys-Val-Lys-Gln-Lys-Tyr-Gly-Gln-Phe- |
| | Pro-Gln-Gly-Thr-Glu-Val-Thr-Tyr-Thr-Cys-Ser-Gly-Asn-Tyr-Phe-Leu-Met |
| (SEQ ID NO: 5) | |
| e. | (S4, Sushi-4) Arg-Ala-Glu-His-Lys-Val-Lys-Lys-Ile-Val-Lys-Gln-Leu-Tyr-Gly- |
| | Gln-Phe-Arg-Gln-Leu-Thr-Arg-Val-Thr-Arg-Thr-Cys-Ser-Arg-Phe-Leu |
| (SEQ ID NO: 6) | |
| f. | (S5, Sushi-5) His-Lys-Val-Lys-Lys-Ile-Val-Lys-Gln-Leu-Tyr-Arg-Ala-Glu-His- |
| | Lys-Val-Lys-Lys-Ile-Val-Lys-Gln-Leu |
| (SEQ ID NO: 7) | |
| g. | (Sushi-6-vg1) Met-Arg-Lys-Leu-Val-Leu-Ala-Leu-Ala-Lys-Ala-Leu-Ala-Lys-Val- |
| | Asp-Lys-Lys-Asn-Leu |
| (SEQ ID NO: 8) | |
| h. | (Sushi-7-vg2) Leu-Leu-Asn-Ala-Val-Pro-His-Lys-Ala-Thr-His-Ala-Ala-Leu-Lys- |
| | Phe-Leu-Lys-Glu-Lys |
| (SEQ ID NO: 9) | |
| i. | (Sushi-8-vg3) Gly-Val-Ser-Thr-Thr-Val-Leu-Asn-Ile-Tyr-Arg-Gly-Ile-Ile-Asn-Leu- |
| | Leu-Gln-Leu-Asn-Val-Lys-Lys |
| (SEQ ID NO: 10) | |
| j. | (Sushi-8-vg3) Ile-Tyr-Arg-Gly-Ile-Ile-Asn-Leu-Ile-Gln-Leu-Ala-Val-Lys-Lys-Ala- |
| | Gln-Asn-Val-Tyr-Gln-Met. |

Two or more of the peptides can be covalently bonded together. A peptide comprising Sushi-3 (SEQ ID NO:3), for example, can be present as a dimer of two sequences bonded together through a disulfide linkage at the Cys²⁷ residue of Sushi-3 (SEQ ID NO:3).

Peptides comprising sequences that occur naturally can be made synthetically or derived from a natural source, such as an organism or a plant, or can be expressed through a nucleic acid that codes for the peptide. Particularly useful methods for deriving the disclosed peptides include those disclosed in U.S. Pat. No. 6,719,973 to Ding et al.

Synthetic peptides and synthetic variations of the naturally occurring sequences can be made by methods known in the art. One method involves linking two or more peptides or polypeptides together using peptide chemistry techniques. For example, peptides can be chemically synthesized using currently available laboratory equipment using either Fmoc (9-fluorenylmethyloxycarbonyl) or Boc (tert-butyloxycarbonoyl) chemistry (Applied Biosystems, Inc., Foster City, Calif.). For example, a peptide can be synthesized and not cleaved from its synthesis resin whereas the other fragment of a peptide can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant, Synthetic Peptides: A User Guide. W.H. Freeman and Co., N.Y. 1992; Bodansky and Trost, Ed. Principles of Peptide Synthesis. Springer-Verlag Inc., N.Y., 1993, which are incorporated herein by this reference at least for their teachings of peptide synthesis.)

The peptides can also include isolated or purified forms of naturally occurring sequences, for example those that are 90%, 95%, or 98% pure. The synthetic peptides can also be pure, for example 90%, 95%, or 98% pure.

The peptides can be salts, for example, acid- or base-addition salts, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, pamoic acid and fumaric acid, or by reaction with an inorganic base such as sodium hydroxide, ammonium hydroxide, potassium hydroxide, and organic bases such as mono-, di-, trialkyl and aryl amines, substituted ethanolamines, and acetates. One specific example of a peptide salt is a peptide comprising the Sushi-3 (SEQ ID NO:3) sequence as an acetate salt.

The peptides can be conjugated to the disclosed lipids in a variety of ways, including conjugation through the carboxy terminus, amino terminus, or through a side-chain of a residue present in the sequence. The amino terminus or an amino containing side-chain, for example, can be conjugated to a lipid through an amide or amine bond. The carboxy terminus or a carboxy containing side-chain can be conjugated to a lipid through an amide or ester bond, for example. An alcohol, thiol, or other reactive moiety in a side-chain of a residue can be conjugated through a variety of bonds, including ether, disulfide, and others. Many of the above groups, e.g., amino, alcohol, carboxylic acid, can also be used to form a suitable leaving group which can react with a nucleophile to provide a conjugate. A variety of methods of conjugation would be readily apparent to one skilled in the art.

As a specific example, a peptide comprising Sushi-3 (SEQ ID NO:3) can be conjugated with a lipid at its carboxy terminus or can be conjugated through a side-chain of a residue in the sequence. For example, a lipid can be conjugated to a peptide comprising Sushi-3 (SEQ ID NO: 3) through the carboxylic acid group (carboxy terminus) of the Met³⁴ residue of Sushi-3 (SEQ ID NO:3). A peptide comprising Sushi-3 (SEQ ID NO:3) can also be conjugated through the Cys²⁷, Ser²⁸ or Tyr³¹ residues.

In another example, the peptide can be conjugate to the lipid by introducing a pre-fabricated lipid-conjugated amino acid derivative to the peptide chain assembly process. As a specific example, the lipid can be conjugated to the Sushi-3 peptide (SEQ ID NO:3) through the Cys²⁷, Ser²⁸, Asn³⁰, or Tyr³¹ residues according to the following process. With reference to FIG. 2, a lipidated amino acid derivate can be introduced as a pre-fabricated building block to the peptide synthesis on a solid support. The conjugated lipopeptide can be obtained after completion of the chain assembly process, cleavage of the protected peptide from the resin and cleavage of all side chain protecting groups. A wide variety of lipid conjugated amino acid derivatives can be used for the Cys²⁷, Ser²⁸ Asn³⁰, or Tyr³¹ or other Sushi-3 (SEQ ID NO:3) residues.

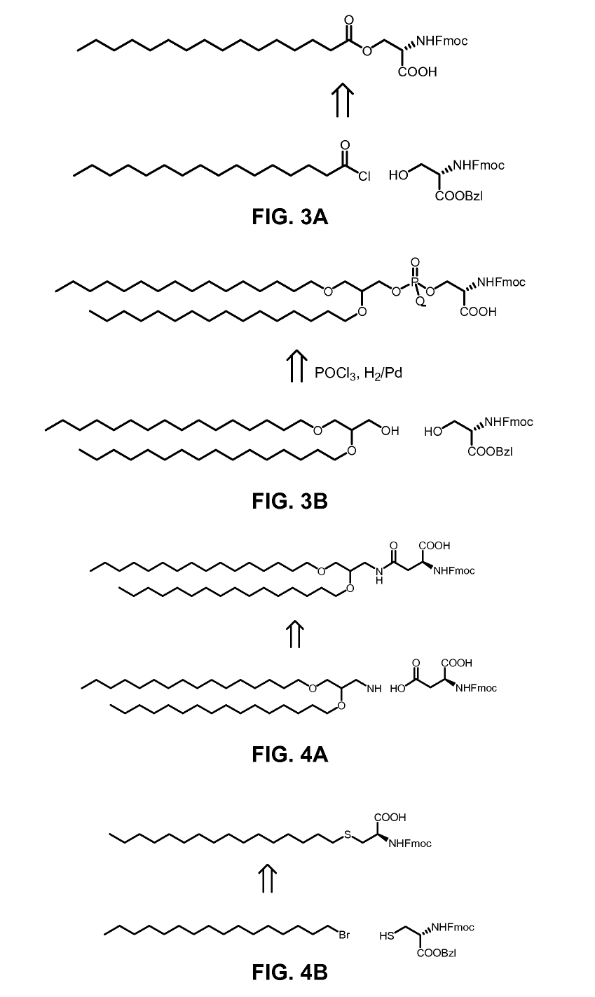

With reference to FIG. 3A and FIG. 3B, one example for a lipidated amino acid derivative is a serine palmic ester (or ether) conjugate which can be produced from Fmoc-Ser-OR and the palmic acid chloride. Similar conjugations can be used for Tyr³¹ of Sushi-3 (SEQ ID NO: 3). Disubstituted phosphoglycerol amino acids (glycerol ethers or esters) can be linked to the hydroxyl functions of serine and tyrosine via reaction with POCl₃.

In another example of a Sushi-3 (SEQ ID NO: 3) peptide-lipid conjugate, aspartic acid derivatives can be used for the synthesis of Asn lipid conjugates by amide bond formation with amino glycerol ethers or esters, as shown in FIG. 4A. The thiol function of the Cys side chain can also be employed in a thioether formation with a wide variety of halogenated carbohydrate structures. One example for Cys thioether is shown in FIG. 4B. A new amino acid could also be used as a substitute residue for Met³⁴ in Sushi-3 (SEQ ID NO: 3).

In a further example, Fmoc-based solid phase synthesis can be used. According to this approach, longer sequences (>10 amino acids) can be produced using a segment condensation approach with additional in-process controls. The 34mer Sushi-3 (SEQ ID NO:3), for example, can be produced from 3 or even 4 peptide segments, such as those listed below.

| (SEQ ID NO: 11) | |
|---|---|
| a. | Segment 1: Boc-His(Trt)-Ala-Glu(OtBu)-His(Trt)-Lys(Boc)-Val-Lys(Boc)-Ile-Gly- |
| | OH |
| (SEQ ID NO: 12) | |
| b. | Segment 2: Fmoc-Val-Glu(OtBu)-Gln(Trt)-Lys(Boc)-Tyr(tBu)-Gly-Gln(Trt)-Phe- |
| | Pro-Gln(Trt)-Gly-OH |
| (SEQ ID NO: 13) | |
| c. | Segment 3: Fmoc-Thr(tBu)-Glu(OtBu)-Val-Thr(tBu)-Tyr(tBu)-Thr(tBu)-Cys(Trt)- |
| | Ser(tBu)-Gly-OH |
| (SEQ ID NO: 14) | |
| d. | Segment 4: Fmoc-Asn(Trt)-Tyr(tBu)-Phe-Leu-Met-OH |

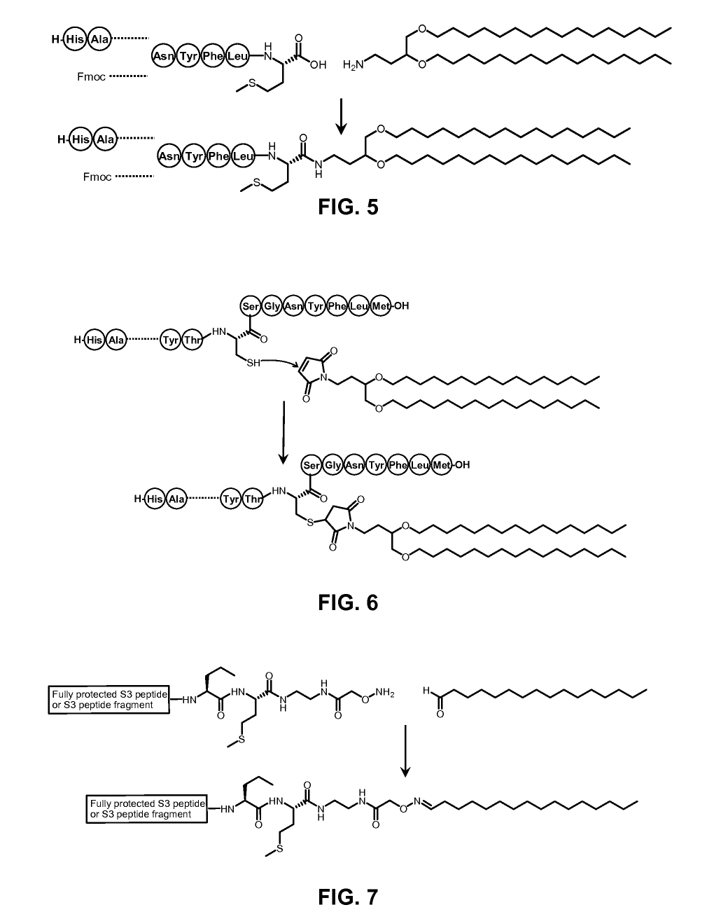

The above four segments, for example, can be produced by standard solid phase peptide synthesis and subsequently condensated to the final Sushi-3 (SEQ ID NO:3) in solution. With reference to FIG. 5, the lipid component can be introduced C-terminally either prior (using Segment 4, for example) or at the end of the condensation cascade (ligation to the fully protected Sushi-3 (SEQ ID NO:3) peptide).

In yet a further example, chemical ligation techniques can be used to ligate the lipid moiety with the native peptide in a regioselective manner. For example, the covalent ligation of the multifunctional Sushi-3 (SEQ ID NO:3) peptide with a lipid component with a maleimide function can be achieved via thioether formation, as shown in FIG. 6. Alternatively, oxime ligation can be used to conjugate native peptides e.g. involving the C-terminal carboxyl function. In this case, the peptide (segment) can be synthesized on special resins (e.g., NovaSyn® TG resin) for direct introduction of hydroxylamine functional groups that react selectively with aldehydes to stable ketoximes, as shown in FIG. 7.

### Lipids

The disclosed lipids include natural and non-natural hydrophobic residues. The lipids can comprise a fatty acid moiety comprising a carboxylic acid that is bonded to a substituted or unsubstituted C₂-C₆₀ alkyl, alkenyl, or alkynyl group that provides sufficient hydrophobic character to the lipid to enhance the interaction of the peptide with LPS. Examples of such lipids include farnesyl-based lipids, geranylgeranyl-based lipids, lauric acid (CH₃(CH₂)₁₀COOH, n-dodecanoic acid), myristic acid (CH₃(CH₂)₁₂COOH, n-tetradecanoic acid), palmitic acid (CH₃(CH₂)₁₄COOH, n-hexadecanoic acid), stearic acid, (CH₃(CH₂)₁₆COOH, n-octadecanoic acid), arachidic acid (CH₃(CH₂)₁₈COOH, n-eicosanoic acid), lignoceric acid (CH₃(CH₂)₂₂COOH, n-tetracosanoic acid), palmitoleic acid (CH₃(CH₂)₅CH═CH(CH₂)₇COOH, cis-9-hexadecenoic acid), oleic acid (CH₃(CH₂)₇CH═CH(CH₂)₇COOH, cis-9-octadecenoic acid), linoleic acid, α-linoleic acid, arachidonic acid, and triacylglycerols.

Other examples of the lipids include phospholipids, glycerophospholipids, glycolipids such as galactolipids, glycerophospholipids (also known as phosphoglycerides), sphingolipids such as ceramide, sphingomyelin, dolichol, glucocerebrosides, globosides and gangliosides.

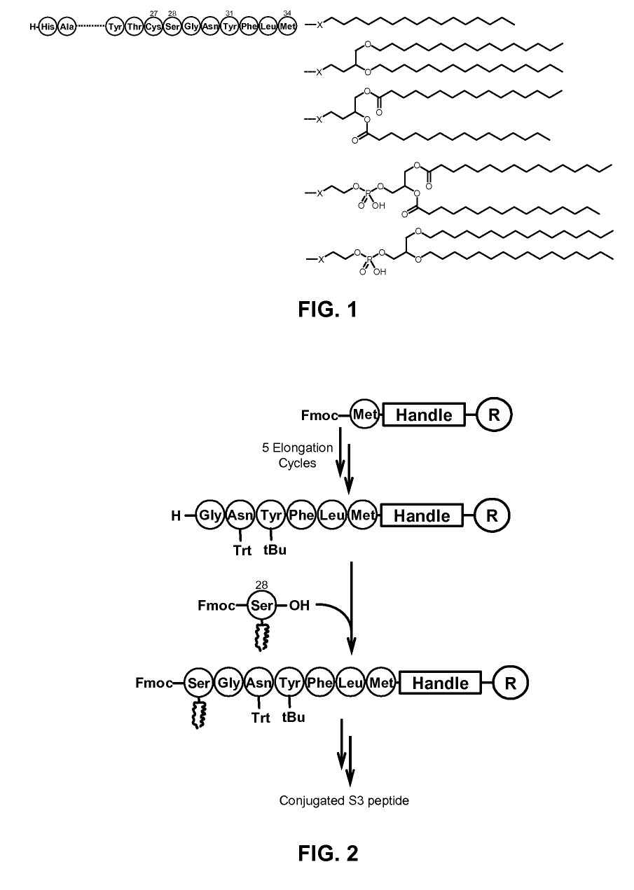

Specific examples of lipids that can be used are shown in FIG. 1, wherein X is a linking group, such as O or NH. As shown in FIG. 1, the exemplary lipids are conjugated to the peptide, which in this example is Sushi-3 (SEQ ID NO:3), at the carboxy terminus of the Met³⁴ residue.

Still further examples of lipids that can be used are fatty acids. By “fatty acid” is meant a carboxylic acid with at least 10 carbon atoms. In one aspect, the fatty acids can comprise at least 10, at least 12, at least 14, at least 16, at least 18, or at least 20 carbon atoms. In some specific examples, the fatty acids can contain 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 carbon atoms, where any of the stated values can form an upper or lower endpoint of a range. In other examples, the fatty acids can comprise a mixture of fatty acids having a range of carbon atoms. For example, the fatty acids can comprise from about 10 to about 40, from about 12 to about 38, from about 14 to about 36, from about 16 to about 34, from about 18 to about 32, or from about 20 to 30 carbon atoms.

The fatty acids suitable for use herein can be saturated, unsaturated, or a mixture of saturated and unsaturated fatty acids. By “saturated” is meant that the molecule or residue contains no carbon-carbon double or triple bounds. By “unsaturated” is meant that the molecule or residue contains at least one carbon-carbon double or triple bond.

The fatty acids that can be used in the disclosed compounds and methods can be derived from any source. Such oils typically contain mixtures of saturated and unsaturated fatty acids, but can be processed to result in a particular mixture of fatty acids (e.g., containing all saturated, all unsaturated, mixtures of both, or mixtures with fatty acids of a certain chain length or range of chain lengths).

Saturated Fatty Acids

Examples of specific saturated fatty acids that are suitable for the compounds and methods disclosed herein include, but are not limited to, capric acid (C10), lauric acid (C12), myristic acid (C14), palmitic acid (C16), margaric acid (C17), stearic acid (C18), arachidic acid (C20), behenic acid (C22), lignoceric acid (C24), cerotic acid (C26), montanic acid (C28), and melissic acid (C30), including branched and substituted derivatives thereof.

Unsaturated Fatty Acids

The unsaturated fatty acids that are suitable for the compounds and methods disclosed herein can comprise at least one unsaturated bond (i.e., a carbon-carbon double or triple bond). In one example, the unsaturated fatty acids can comprise at least 2, at least 3, at least 4, at least 5, at least 6, at least 7, at least 8, at least 9, or at least 10 carbon-carbon double bonds, triple bonds, or any combination thereof. In another example, the unsaturated fatty acids can comprise 1, 2, 3, 4, 5, 6, 7, 8, 9, or 10 unsaturated bonds, where any of the stated values can form an upper or lower endpoint of a range.

Monoene Acids and Residues

In one aspect, the unsaturated fatty acids can comprise one carbon-carbon double bond (i.e., a monoene acid or residue). Examples of unsaturated fatty acids that are suitable for the compounds and methods disclosed herein include, but are not limited to, those in the following Table 2.

TABLE 2
Examples of Monoene

| Total number of carbon | Carbon number where double bond begins. |
|---|---|
| atoms in the fatty acid | (“c” denotes a cis double bond; |
| or residue chain. | “t” denotes a trans double bond) |
| 10 | 4c |
| 12 | 4c |
| 14 | 4c and 9c |
| 16 | 3t, 4c, 5t, 6c, 6t, 9c (palmitooleic), and 11c |
| 18 | 3t, 5c, 5t, 6c (petroselinic), 6t, 9c (oleic), 10c, |
| | 11c (cis-vaccenic), 11t (vaccenic), and 13c |
| 20 | 5c, 9c (gadolenic), 11c, 13c, and 15c |
| 22 | 5c, 11c (cetoleic), 13c (erucic), and 15c |
| 24 | 15c (selacholeic, nervonic) |
| 26 | 9c, and 17c (ximenic) |
| 28 | 9c, 19c (lumequic) |
| 30 | 21c |

Polyene Acids and Residues (Methylene Interrupted)

In another aspect, the unsaturated fatty acids can comprise at least two unsaturated bonds (e.g., polyene acids or residues). In some examples, the unsaturated fatty acids can comprise at least one pair of methylene interrupted unsaturated bonds. By “methylene interrupted unsaturated bond” is meant that one carbon-carbon double or triple bond is separated from another carbon-carbon double or triple bond by at least one methylene group (i.e., CH₂). Specific examples of unsaturated fatty acids that contain at least one pair of methylene interrupted unsaturated bonds include, but are not limited to, the n-1 family derived from 9, 12, 15-16:3; n-2 family derived from 9, 12, 15-17:3, 15:3, 17:3, 17:4, 20:4; n-3 family derived from 9, 12, 15-18:3, 15:2, 15:3, 15:4, 16:3, 16:4, 18:3 (α-linolenic), 18:4, 18:5, 20:2, 20:3, 20:4; 20:5 (EPA), 21:5, 22:3, 22:5 (DPA), 22:6 (DHA), 24:3, 24:4, 24:5, 24:6, 26:5, 26:6, 28:7, 30:5; n-4 family derived from 9, 12-16:2, 16:2, 16:3, 18:2, 18:3; n-5 family derived from 9, 12-17:2, 15:2, 17:2, 17:3, 19:2, 19:4, 20:3, 20:4 21:4, 21:5; n-6 family derived from 9, 12-18:2, 15:2, 16:2, 18:2 (linoleic acid), 18:3 (γ-linolenic acid); 20:2, 20:3, 20:4 (arachidonic acid), 22:2, 22:3, 22:4 (adrenic acid), 22:5, 24:2, 24:4, 25:2, 26:2, 30:4; n-7 family derived from 9-16:1, 15:2, 16:2, 17:2, 18:2, 19:2; n-8 family derived from 9-17:1, 15:2, 16:2, 17:2, 18:2, 19:2; n-9 family derived from 9-18:1, 17:2, 18:2, 20:2, 20:3, 22:3, 22:4; n-11 family 19:2, and the n-12 family 20:2.

In the above paragraph, the compounds are identified by referring first to the “n-x family,” where x is the position in the fatty acid where the first double bond begins. The numbering scheme begins at the terminal end of the fatty acid, where, for example, the terminal CH₃ group is designated position 1. In this sense, the n-3 family would be an omega-3 fatty acid, as described herein. The next number identifies the total number of carbon atoms in the fatty acid. The third number, which is after the colon, designates the total number of double bonds in the fatty acid. So, for example, in the n-1 family, 16:3, refers to a 16 carbon long fatty acid with 3 double bonds, each separated by a methylene, wherein the first double bond begins at position 1, i.e., the terminal end of the fatty acid. In another example, in the n-6 family, 18:3, refers to an 18 carbon long fatty acid with 3 methylene separated double bonds beginning at position 6, i.e., the sixth carbon from the terminal end of the fatty acid, and so forth.

Some other examples are fatty acids that contain at least one pair of unsaturated bonds interrupted by more than one methylene group. Suitable examples of these acids and residues thereof include, but are not limited to, those in the following Table 3:

TABLE 3
Examples of Polyene Acids and Residues with Double
Bonds Interrupted by Several Methylene Units

| Total number of carbon | Carbon number where double bond begins. |
|---|---|
| atoms in the fatty acid | (“c” denotes a cis double bond; |
| or residue chain. | “t” denotes a trans double bond) |
| 18 | 5, 9 |
| | 5, 11 |
| | 2t, 9, 12 |
| | 3t, 9, 12 |
| | 5t, 9, 12 |
| | 5, 9, 12 |
| | 5, 11, 14 |
| | 3t, 9, 12, 15 |
| | 5, 9, 12, 15 |
| 20 | 5, 11 |
| | 5, 13 |
| | 7, 11 |
| | 7, 13 |
| | 5, 11, 14 |
| | 7, 11, 14 |
| | 5, 11, 14, 17 |
| 22 | 5, 11 |
| | 5, 13 |
| | 7, 13 |
| | 7, 15 |
| | 7, 17 |
| | 9, 13 |
| | 9, 15 |

Polyene Acids and Residues (Conjugated)

Still other examples of unsaturated fatty acids that are suitable for use in the compounds and methods disclosed herein are those that contain at least one conjugated unsaturated bond. By “conjugated unsaturated bond” is meant that at least one pair of carbon-carbon double and/or triple bonds are bonded together, without a methylene (CH₂) group between them (e.g., —CH═CH—CH═CH—). Specific examples of unsaturated fatty acids that contain conjugated unsaturated bonds include, but are not limited to, those in the following Table 4.

TABLE 4
Examples of Conjugated Polyene Acids and Residues

| Total number of carbon | Carbon number where double bond begins. |
|---|---|
| atoms in the fatty acid | (“c” denotes a cis double bond; |
| or residue chain. | “t” denotes a trans double bond) |
| 10 | 2t, 4t, 6c |
| | 2c, 4t, 6t |
| | 3t, 5t, 7c |
| | 3c, 5t, 7t |
| 12 | 3, 5, 7, 9, 11 |
| 14 | 3, 5, 7, 9, 11 |
| 18 | 10t, 12t |
| | 8c, 10t, 12c (jacaric) |
| | 8t, 10t, 12c (calendic) |
| | 8t, 10t, 12t |
| | 9t, 11t, 13c (catalpic) |
| | 9c, 11t, 13t (α-eleostearic) |
| | 9c, 11t, 13c (punicic) |
| | 9t, 11t, 13t (β-eleostearic) |
| | 9c, 11t, 13t, 15c (α-parinaric) |
| | 9t, 11t, 13t, 15t (β-parinaric) |

Exemplary Unsaturated Fatty Acids

Some specific examples of unsaturated fatty acids that can be used in the compounds and methods disclosed herein include, but are not limited to linoleic acid, linolenic acid, γ-linolenic acid, arachidonic acid, mead acid, stearidonic acid, α-eleostearic acid, eleostearic acid, pinolenic acid, docosadienic acid, docosatetraenoic acid, docosapentaenoic acid, docosahexaenoic acid, octadecadienoic acid, octadecatrienoic acid, eicosatetraenoic acid, eicosapentaenoic, or any combination thereof. In one aspect, the unsaturated fatty acid residue can be derived from eicosapentaenoic acid 20:5ω3 (EPA), docosahexaenoic acid 22:6ω3 (DHA), docosapentaenoic acid 22:5ω3 (DPA), and any combination thereof.

Unsaturated Fatty Acids with Triple Bonds

Additional examples of suitable unsaturated fatty acids which are suitable in the disclosed compounds and methods include, but are not limited to, allenic and acetylenic acids, such as, C14: 2, 4, 5; C18: 5, 6 (laballenic); 5, 6, 16 (lamenallenic); C18: 6a (tarinic); 9a; 9a, 11t (ximenynic); 9a, 11a; 9a, 11a, 13c (bolekic); 9a, 11a, 13a, 15e, 8a, 10t (pyrulic) 9c, 12a (crepenynic); 9c, 12a, 14c (dehydrocrepenynic acid); 6a, 9c, 12c; 6a, 9c, 12c, 15c, 8a, 11c, 14c and corresponding Δ17e derivatives, 8-OH derivatives, and Δ17e, 8-OH derivatives.

Additional Fatty Acids

Branched-chain acids, particularly iso-acids and anteiso acids, polymethyl branched acids, phytol based acids (e.g., phytanic, pristanic), furanoid acids are also suitable fatty acids, including the residues derived therefrom, for use in the compounds and methods disclosed herein.

Still further, suitable fatty acids include, but are not limited to, cyclic acids, such as cyclopropane fatty acids, cyclopropene acids (e.g., lactobacillic), sterulic, malvalic, sterculynic, 2-hydroxysterculic, aleprolic, alepramic, aleprestic, aleprylic alepric, hydnocarpic, chaulmoogric hormelic, manaoic, gorlic, oncobic, cyclopentenyl acids, and cyclohexylalkanoic acids.

Hydroxy acids, particularly butolic, ricinoleic, isoricinoleic, densipolic, lesquerolic, and auriolic are also suitable fatty acids that can be used in the compounds and methods disclosed herein.

Epoxy acids, particularly epoxidated C18:1 and C18:2, and furanoid acids are further examples of fatty acids that can be used in the disclosed compounds and methods.

### Conjugates

The disclosed compositions are conjugates of the peptides and lipids disclosed herein. For example, the disclosed conjugates can be represented by the following formula, Formula IA-C

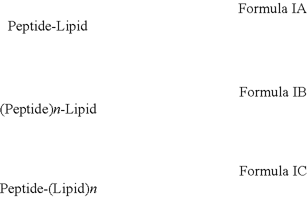

where n is an integer of from 1 to 20; “Peptide” is any of the peptides disclosed herein, including SEQ ID NOs: 1-15, and variants and fragments thereof; and “Lipid” is any lipid molecule disclosed herein.

These peptide-lipid conjugates can be characterized as a peptide having one or more lipid moieties covalently linked to the peptide. There are various examples of linking a lipid to a peptide disclosed herein. For example, a lipid can be linked to the C-terminus of the peptide. In another example, a lipid can be linked to the N-terminus of the peptide. Still further, a lipid can be linked to a side chain of one or more amino acid residues of the peptide. Even further, lipids can be linked at more than one of these locations on the peptide chain.

One can use standard chemistry coupling techniques to link a lipid to a peptide as disclosed herein.

Linker

Still further, the lipids disclosed herein can be linked to a peptide as disclosed herein with a Linker. For example, the disclosed conjugates can be represented by the following formula, Formula IIA-B

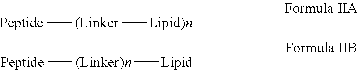

where n is an integer of from 1 to 20; “Peptide” is any of the peptides disclosed herein, including SEQ ID NOs: 1-15, and variants and fragments thereof; and “Lipid” is any lipid molecule disclosed herein.

The Linker can be a moiety that spans from 1 to 6 atoms. For example, the Linker can span from 2 to 5 atoms, from 3 to 4 atoms, from 1 to 3 atoms, from 4 to 6 atoms, or from 3 to 5 atoms. Some specific examples of Linkers include, but are not limited to, —O— (i.e., an ether), —S— (i.e., a thioether), methylhydrazine, methylhydrazone, methylenepropyl, butyl, pentyl, 1,3 substituted cyclopentyl, 1,3-substituted cyclohexyl, 1,3-substituted cycloheptyl, 1,4-substituted cyclohexyl, 1,4-substituted cycloheptyl, ethyoxyl, propoxyl, butoxyl, methoxymethyl, methoxyethyl, methoxypropyl, ethoxymethyl, ethoxyethyl, propoxymethyl, methylaminomethyl, methylaminoethyl, methylaminopropyl, ethylaminomethyl, ethylaminoethyl, propylaminomethyl, methoxymethoxymethyl, methoxymethoxyethyl, —C(O)OCH₂—, —C(O)OCH₂CH₂—, —C(O)OCH₂CH₂CH₂—, —CH₂C(O)O—, —CH₂C(O)OCH₂—, or —CH₂CH₂C(O)OCH₂—, including pharmaceutically acceptable salt thereof.

In many examples, the Linker can be a liner moiety. Some specific examples of linear linkers are shown in the following table. The bonds from the Linker to the peptide and lipid moieties are omitted for clarity.

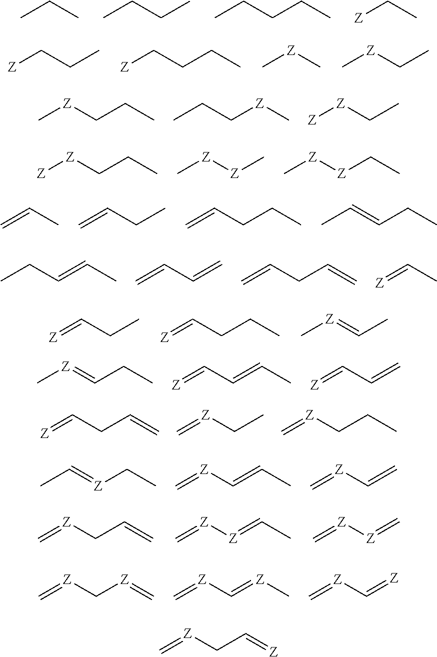

wherein each Z is, independent of the others, O, C(O), S, SO, SO₂, N, or NH, and with the understanding that the valences of each Z are not violated in the linker. Pharmaceutically acceptable salt of these moieties are also contemplated.

In other examples, the Linker can be an aryl moiety. Some specific examples of aryl linkers are shown in the following table. The bonds from the Linker to the peptide and lipid moieties are omitted for clarity but can be in either the ortho, meta, or para configuration.

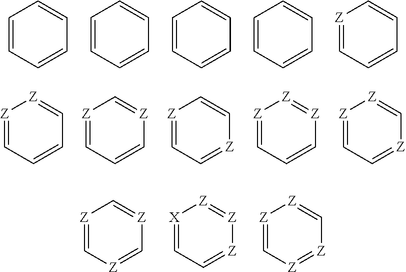

wherein Z is N and with the understanding that the valences of each Z are not violated in the Linker. Pharmaceutically acceptable salt of these moieties are also contemplated.

In other examples, the Linker can be a cyclic moiety. Some specific examples of linear Linkers are shown in the following table. Again, the bonds from the Linker to the Ar¹ and Ar² moieties are omitted for clarity but can be in either the ortho, meta, or para configuration in the case of six membered rings or 1,2-, 1,3-, 1,4-, or 1,5- arrangement in the case of five membered rings.

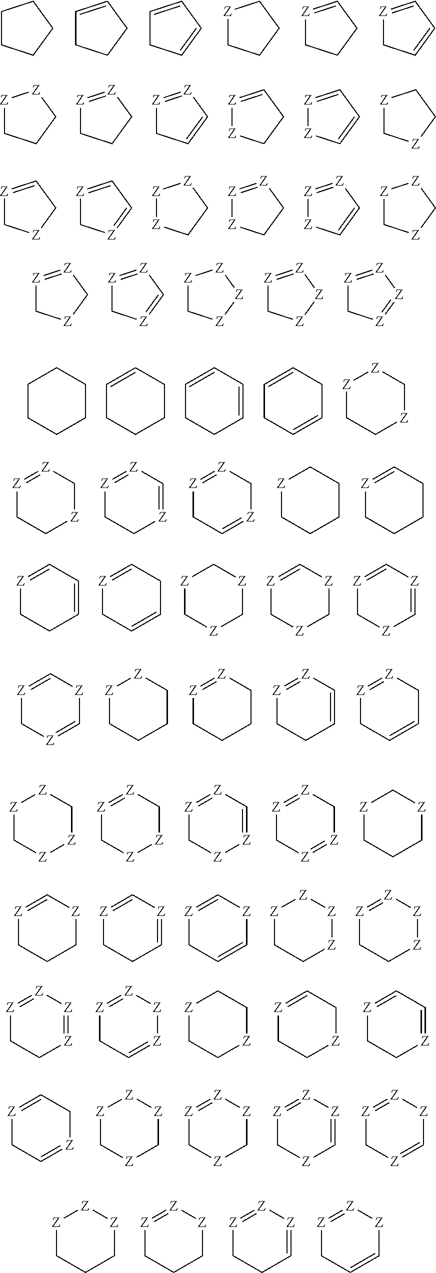

wherein each Z is, independent of the others, O, S, N, or NH, NR², and with the understanding that the valences of each Z are not violated in the linker and where R² is a C₁-C₆ alkyl or C₁-C₆ acyl, ac. Pharmaceutically acceptable salt of these moieties are also contemplated.

In further examples, the linker between the peptide and the lipid can comprise a substituted or unsubstituted alkyl, cycloalkyl, cycloheteroalkyl, alkoxy, alkenyl, cycloalkenyl, cycloheteroalkenyl, alkynyl, cycloalkynyl, cycloheteroalkynyl, aryl, heteroaryl, carboxylate, carbonate, ether, polyether, ester, polyester, amine, polyamine, polyalkylene, silyl, dendrimeric polyamine, or dendrimeric polyol.

The Linker can, in further specific examples, comprise a substituted or unsubstituted C₁-C₆ branched or straight-chain alkyl, such as methyl, ethyl, n-propyl, isopropyl, n-butyl, isobutyl, sec-butyl, tert-butyl, n-pentyl, isopentyl, neopentyl, sec-pentyl, or hexyl. In a specific example, the Linker can comprise a polyalkylene (i.e., —(CH₂)ₙ—, wherein n is from 1 to 25, from 1 to 20, from 1 to 15, from 1 to 10, from 1 to 5, from 1 to 3, or from 10 to 20). Still further, the Linker can comprise a cycloalkyl, such as cyclopentyl, cyclohexyl, or cyclopropyl. The Linker can also be a cycloheteroalkyl like piperazine, 2-methylpiperazine, 1,3-di(piperidin-4-yl)propane, pyranyl, and the like.

In another example, the Linker can comprise a C₁-C₆ branched or straight-chain alkoxy such as a methoxy, ethoxy, n-propoxy, isopropoxy, n-butoxy, isobutoxy, sec-butoxy, tert-butoxy, n-pentoxy, isopentoxy, neopentoxy, sec-pentoxy, or hexoxy. In still other examples, the Linker can comprise a C₂-C₆ branched or straight-chain alkyl, wherein one or more of the carbon atoms are substituted with oxygen (e.g., an ether), sulfur (e.g., a thioether), or nitrogen (e.g., an amino). For example, a suitable Linker can include, but is not limited to, a methoxymethyl, methoxyethyl, methoxypropyl, methoxybutyl, ethoxymethyl, ethoxyethyl, ethoxypropyl, propoxymethyl, propoxyethyl, methylaminomethyl, methylaminoethyl, methylaminopropyl, methylaminobutyl, ethylaminomethyl, ethylaminoethyl, ethylaminopropyl, propylaminomethyl, propylaminoethyl, methoxymethoxymethyl, ethoxymethoxymethyl, methoxyethoxymethyl, methoxymethoxyethyl, and the like, and derivatives thereof.

In one specific example, the Linker can be —CH₂CH₂NR²CH₂CH₂—, or —CH₂CH₂CHR²CH₂CH₂—, where R² is —CH₂CH₂NHSO₂-p-CH₃-Ph (Ph is phenyl).

It is also possible to converted the lipid and/or peptides into more reactive, activated esters by a carbodiimide coupling with a suitable alcohol, e.g., 4-sulfo-2,3,5,6-tetrafluorophenol, N-hydroxysuccinimide or N-hydroxysulfosuccinimide. This results in a more reactive, water-soluble activated ester moiety. Various other activating reagents that can be used for the coupling reaction include, but are not limited to, 1-ethyl-3-(3-dimethylaminopropyl)carbodiimide (EDC), dicyclohexylcarbodiimide (DCC), N,N′-diisopropyl-carbodiimide (DIP), benzotriazol-1-yl-oxy-tris-(dimethylamino)phosphonium hexa-fluorophosphate (BOP), hydroxybenzotriazole (HOBt), and N-methylmorpholine (NMM), including mixtures thereof.

### Encapsulated Peptide-Lipid Conjugates

The peptide-lipid conjugates disclosed herein can be formulated in drug delivery systems, such as by encapsulation in a polymeric matrix such as a polymeric matrix of an implant, a microparticle, a fiber, or otherwise encapsulated. Encapsulated lipid-peptide conjugates can have a variety of uses, for example in pharmaceutical formulations that can be delivered to a subject. The microparticle or implant can modulate the release of the peptide-lipid conjugate, e.g., extend the release of peptide-lipid for days, months or years following a single administration. For some applications, including pharmaceutical formulations, the microparticle can enhance the ability of the peptide-lipid conjugate by shielding the peptide-lipid conjugate from interfering biological substances until the peptide-lipid conjugate can reach lipopolysaccharide (LPS).

In one example, the peptide-lipid conjugates can be encapsulated in a microparticle. The microparticles can include nanoparticles, microspheres, nanospheres, microcapsules, nanocapsules, and particles, in general. The microparticles can have sizes in the range of about 10 nanometers (nm) to about 2 mm (millimeters). For example, the microparticles can have an average or mean particle size of from about 20 μm to about 125 μm. In one example the range of mean particle size is from about 40 μm to about 90 μm. In another example the range of mean particle sizes is from about 50 μm to about 80 μm.

Any suitable amount of the peptide-lipid conjugate can be encapsulated in a microparticle, including 1%, 5%, 10%, 20%, 30%, 40%, 50%, 60%, 70%, 80%, 90%, 95% by weight peptide-lipid conjugate, relative to the weight of the microparticle.

The microparticles can comprise a variety of biocompatible, biodegradable and non-biodegradable polymers that can be selected by one of skill in the art, such as polyesters. Exemplary microparticles are those comprising poly(lactide), including all racemic forms of lactide (i.e., D-, L-, and D,L-), poly(glycolide), poly(caprolactone), copolymers, terpolymers, and mixtures thereof, such as poly(lactide)-co-(glycolide).

One specific example of a microparticle is one comprising the Sushi-3 (SEQ ID NO:3) sequence conjugated to a lipid that is encapsulated in a poly(lactide)-co-(glycolide) microparticle.

The peptide-lipid conjugates can be encapsulated in microparticles, and the microparticles can be formulated using methods known in the art, including, for example, those methods disclosed in U.S. Patent Publication No. 2007/0190154 and U.S. Pat. No. 5,407,609, both of which are incorporated herein in their entirety by this reference for teachings of microparticle preparation methods.

The peptide-lipid conjugates can be co-processed with a microparticle formulation to enable encapsulation of the peptide-lipid conjugate. For example, an emulsion-based, solvent-extraction microencapsulation process can be used. In this example, an organic solution of polymer and peptide can be prepared. The peptide-lipid conjugate can be dissolved in an aqueous buffer or dissolved or dispersed in an organic phase containing the polymer. The resulting polymer/peptide-conjugate mixture (the dispersed phase) can then be introduced into an inline homogenizer along with an aqueous phase comprising water and surfactant (the continuous phase). An oil-in-water emulsion then exits the homogenizer and enters a chamber of water, which quickly extracts the organic polymer solvent from the emulsion microdroplets that are present. The solvent removal step quickly precipitates the polymer and results in the encapsulation of the peptide-lipid conjugate.

The lipid component of the peptide-lipid conjugate can enhance the ability to encapsulate the peptide by making the peptide-lipid conjugate organically soluble. This can allow for better distribution of the peptide-lipid conjugate in the microparticle. The decreased water solubility of the peptide-lipid conjugate, provided by the lipid, can also increase encapsulation efficiency because the peptide-lipid conjugate will be less soluble in the aqueous continuous phase of the process. Thus, higher loadings can be achieved with the peptide-lipid conjugates, relative to loadings achieved with the peptide itself. The hydrophobic character of the peptide-lipid conjugate can also enhance the release of the conjugate from the microparticle, including enhancements in reduced burst and desired extended-release properties and ability to administer more peptide-lipid through higher loadings of the peptide-lipid conjugate.

By conjugating selected lipids to the peptide, the physical and chemical properties of the peptide-lipid can be modified to match and optimize preparation and performance of the peptide-lipid conjugate in a broad range of drug delivery systems. When liposomes are targeted, for example, the addition of the lipid to the peptide allows for the peptide-lipid conjugate to incorporate into the lipid membranes of liposome. This incorporation improves peptide-lipid conjugate loading and overall liposome performance.

### Methods of Use

The peptide-lipid conjugates disclosed herein have a variety of uses. The conjugates can be used to detect and/or remove LPS in a biological sample that contains LPS by contacting the sample with the peptide-lipid conjugate or a composition or formulation comprising the conjugate. For such detection uses, the peptide-lipid conjugates can also comprise a dye or a label that allows the location of the peptide-lipid conjugate to be identified in a sample.

The peptide-lipid conjugates can also be used as therapeutic agents to treat a number of disorders including a variety of bacterial infections, particularly those associated with Gram-negative bacteria. Other therapeutic uses include the use of the peptide-lipid conjugates for the treatment of sepsis, wound healing, pulmonary diseases, cystic fibrosis, liver failure, and eye diseases.

For therapeutic uses, pharmaceutical compositions and formulations can contain the peptide-lipid conjugates in an effective amount for treating the disorder. The specific effective amount for any particular subject will depend upon a variety of factors including the disorder being treated and the severity of the disorder; the identity and activity of the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts. For example, it is well within the skill of the art to start doses of a composition at levels lower than those required to achieve the desired therapeutic effect and to gradually increase the dosage until the desired effect is achieved. One can also evaluate the particular aspects of the medical history, signs, symptoms, and objective laboratory tests that are known to be useful in evaluating the status of a subject in need of attention for the treatment of ischemia-reperfusion injury, trauma, drug/toxicant induced injury, neurodegenerative disease, cancer, or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: 1) a subject's physical condition is shown to be improved (e.g., a bacterial infection that has decreasing toxic effects), 2) the progression of the disease or condition is shown to be stabilized, or slowed, or reversed, or 3) the need for other medications for treating the disease or condition is lessened or obviated, then a particular treatment regimen will be considered efficacious. If desired, the effective daily dose can be divided into multiple doses for purposes of administration. Consequently, single dose compositions can contain such amounts or submultiples thereof to make up the daily dose.

An effective amount of the peptide-lipid conjugate can also be determined by preparing a series of compositions comprising varying amounts of the peptide-lipid conjugates and determining the release characteristics in vivo and in vitro and matching these characteristics with specific pharmaceutical delivery needs, inter alia, subject body weight, disease condition and the like.

The dosage for the compositions can be adjusted by the individual physician or the subject in the event of any counterindications. Dosage can vary, and can be administered in one or more dose administrations daily, for one or several days. Guidance can be found in the literature for appropriate dosages for given classes of pharmaceutical products.

The pharmaceutical formulations can comprise the peptide-lipid conjugate disclosed herein. In some examples, the peptide-lipid conjugate can be encapsulated in a microparticle, as discussed above. The pharmaceutical formulations can also contain other bioactive agents or drugs, as desired or needed. Generally, the pharmaceutical formulations can comprise the active ingredient(s), such as the peptide-lipid conjugate along with a pharmaceutically acceptable carrier. In some examples, the disclosed microparticles can themselves be pharmaceutically acceptable carriers. The pharmaceutical formulations disclosed herein can be used therapeutically or prophylactically.

A pharmaceutically acceptable carrier or substance can be a material that is not biologically or otherwise undesirable, i.e., the material may be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy, 21^{st} Ed., Lippincott Williams & Wilkins, Philadelphia, Pa., 2005, which is incorporated by reference herein for its teachings of carriers and pharmaceutical formulations. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8 (e.g., from about 7 to about 7.5). Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, anti-inflammatory agents, anesthetics, and the like.

The pharmaceutical formulation can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, marine oils, and injectable organic esters such as ethyl oleate. Aqueous carriers include water, alcoholic/aqueous solutions, and emulsions or suspensions, including saline and buffered media. Parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations for topical administration may include ointments, lotions, creams, gels, drops, suppositories, sprays, liquids and powders. Conventional pharmaceutical carriers, aqueous, powder or oily bases, thickeners and the like can be desirable.

Pharmaceutical formulations for oral administration include, but are not limited to, powders or granules, suspensions or solutions in water or non-aqueous media, capsules, gel-caps, sachets, or tablets. Thickeners, flavorings, diluents, emulsifiers, dispersing aids, or binders can be desirable.

Some of the formulations can potentially be administered as a pharmaceutically acceptable acid- or base-addition salts, as discussed above, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, pamoic acid and fumaric acid, or by reaction with an inorganic base such as sodium hydroxide, ammonium hydroxide, potassium hydroxide, and organic bases such as mono-, di-, trialkyl and aryl amines, substituted ethanolamines, and acetates.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

In one aspect disclosed herein are peptide-lipid conjugates comprising a peptide conjugated to a lipid, wherein the peptide comprises a sequence selected from Sushi-1 (SEQ ID NO:1), SΔ1 (SEQ ID NO:2), Sushi-3 (SEQ ID NO:3), SΔ3 (SEQ ID NO:4), Sushi-4 (SEQ ID NO:5), Sushi-5 SEQ ID NO:6), Sushi-6-vg1 (SEQ ID NO:7), Sushi-7-vg2 (SEQ ID NO:8), Sushi-8-vg3 (SEQ ID NO:9), Sushi-8-vg3 (SEQ ID NO:10), SEQ ID NO:11, SEQ ID NO:12, SEQ ID NO:13, and SEQ ID NO:14.

In further aspect, the peptide-lipid conjugates can comprises Sushi-3 (SEQ ID NO:3).

Also disclosed herein are peptide-lipid conjugates of any preceding aspect, wherein the peptide-lipid conjugate is encapsulated in a polymer matrix.

Also disclosed herein are peptide-lipid conjugates of any preceding aspect, wherein the peptide-lipid conjugate is encapsulated in an implant.

Also disclosed herein are peptide-lipid conjugates of any preceding aspect, wherein the peptide-lipid conjugate is encapsulated in a microparticle.

Also disclosed herein are peptide-lipid conjugates of any preceding aspect, wherein the microparticle comprises poly(lactide), poly(glycolide), or poly(lactide)-co-(glycolide).

Also disclosed herein are peptide-lipid conjugates of any preceding aspect, wherein the peptide-lipid conjugate is encapsulated in a fiber.

Also disclosed herein are peptide-lipid conjugates of any preceding aspect, wherein the peptide-lipid conjugate is encapsulated in a liposome.

Also disclosed herein are peptide-lipid conjugates of any preceding aspect, wherein the peptide is conjugate to the lipid at the carboxy terminus of the peptide.

Also disclosed herein are pharmaceutical compositions comprising a peptide-lipid conjugates of any preceding aspect and a pharmaceutically acceptable carrier.

In another aspect, disclosed herein are the pharmaceutical composition of any preceding aspect, wherein the pharmaceutically acceptable carrier comprises a microparticle.

In another aspect, disclosed herein are methods for treating a disorder associated with lipopolysaccharide (LPS), comprising administering to a subject diagnosed with the disorder associated with LPS a peptide-lipid conjugate comprising a peptide that binds to LPS, in an effective amount to treat the disorder.

In another aspect, the disclosed methods for treating a disorder associated with lipopolysaccharide (LPS) can further comprise selecting a peptide that binds to LPS and conjugating the peptide with a lipid to form the peptide-lipid conjugate.

Also disclosed are the methods for treating a disorder associated with lipopolysaccharide (LPS), wherein the peptide comprises a sequence selected from Sushi-1 (SEQ ID NO:1), SΔ1 (SEQ ID NO:2), Sushi-3 (SEQ ID NO:3), SΔ3 (SEQ ID NO:4), Sushi-4 (SEQ ID NO: 5), Sushi-5 SEQ ID NO: 6), Sushi-6-vg1 (SEQ ID NO: 7), Sushi-7-vg2 (SEQ ID NO:8), Sushi-8-vg3 (SEQ ID NO:9), Sushi-8-vg3 (SEQ ID NO:10), SEQ ID NO:11, SEQ ID NO:12, SEQ ID NO:13, and SEQ ID NO:14.

Also disclosed are the methods for treating a disorder associated with lipopolysaccharide (LPS) of any preceding aspect, wherein the peptide comprises Sushi-3 (SEQ ID NO:3).

Also disclosed are the methods for treating a disorder associated with lipopolysaccharide (LPS) of any preceding aspect, wherein the peptide-lipid conjugate is encapsulated in a polymer matrix.

Also disclosed are the methods for treating a disorder associated with lipopolysaccharide (LPS) of any preceding aspect, wherein the peptide-lipid conjugate is encapsulated in an implant.

Also disclosed are the methods for treating a disorder associated with lipopolysaccharide (LPS) of any preceding aspect, wherein the peptide-lipid conjugate is encapsulated in a microparticle.

Also disclosed are the methods for treating a disorder associated with lipopolysaccharide (LPS) of any preceding aspect, wherein the microparticle comprises poly(lactide), poly(glycolide), or poly(lactide)-co-(glycolide).

Also disclosed are the methods for treating a disorder associated with lipopolysaccharide (LPS) of any preceding aspect, wherein the peptide-lipid conjugate is encapsulated in a fiber.

Also disclosed are the methods for treating a disorder associated with lipopolysaccharide (LPS) of any preceding aspect, wherein the peptide-lipid conjugate is encapsulated in a liposome.

Also disclosed are the methods for treating a disorder associated with lipopolysaccharide (LPS) of any preceding aspect, wherein the peptide is conjugated to the lipid at the carboxy terminus of the peptide.

Also disclosed are the methods for treating a disorder associated with lipopolysaccharide (LPS) of any preceding aspect, wherein the disorder is a bacterial infection.

Also disclosed are the methods for treating a disorder associated with lipopolysaccharide (LPS) of any preceding aspect, wherein the peptide-lipid conjugate is present in a pharmaceutical composition comprising a pharmaceutically acceptable carrier.

In another aspect, disclosed herein are methods for delivering a conjugate, comprising administering to a subject a polymer matrix having encapsulated therein a peptide-lipid conjugate comprising a peptide that binds to lipopolysaccharide (LPS), under conditions effective to deliver the peptide-lipid conjugate to lipopolysaccharide in the subject.

In another aspect, the disclosed methods for delivering a conjugate of any preceding aspect further comprise selecting a peptide that binds to lipopolysaccharide (LPS) and conjugating the peptide with a lipid to form the peptide-lipid conjugate.

Also disclosed are the methods for delivering a conjugate of any preceding aspect, wherein the peptide comprises a sequence selected from Sushi-1 (SEQ ID NO:1), SΔ1 (SEQ ID NO:2), Sushi-3 (SEQ ID NO:3), SΔ3 (SEQ ID NO:4), Sushi-4 (SEQ ID NO: 5), Sushi-5 SEQ ID NO: 6), Sushi-6-vg1 (SEQ ID NO: 7), Sushi-7-vg2 (SEQ ID NO:8), Sushi-8-vg3 (SEQ ID NO:9), Sushi-8-vg3 (SEQ ID NO:10); SEQ ID NO:11, SEQ ID NO:12, SEQ ID NO:13, and SEQ ID NO:14.

Also disclosed are the methods for delivering a conjugate of any preceding aspect, wherein the peptide comprises Sushi-3 (SEQ ID NO:3).

Also disclosed are the methods for delivering a conjugate of any preceding aspect, wherein the polymer matrix is a polymer matrix of an implant.

Also disclosed are the methods for delivering a conjugate of any preceding aspect, wherein the polymer matrix is a polymer matrix of a microparticle.

Also disclosed are the methods for delivering a conjugate of any preceding aspect, wherein the microparticle comprises poly(lactide), poly(glycolide), or poly(lactide)-co-(glycolide).

Also disclosed are the methods for delivering a conjugate of any preceding aspect, wherein the polymer matrix is a polymer matrix of a fiber.

Also disclosed are the methods for delivering a conjugate of any preceding aspect, wherein the peptide is conjugated to the lipid at the carboxy terminus of the peptide

Sequence Listing

SEQ ID NO: 1 (S1, Sushi-1) Gly-Phe-Lys-Leu-Lys-Gly-Met-Ala-Arg-Ile-Ser-Cys-Leu-Pro-
Asn-Gly-Gln-Trp-Ser-Asn-Phe-Pro-Pro-Lys-Cys-Ile-Arg-Glu-Cys-Ala-Met-Val-Ser-Ser
SEQ ID NO: 2 (SΔ1) Gly-Phe-Lys-Leu-Lys-Gly-Lys-Ala-Lys-Ile-Ser-Cys-Leu-Pro-Asn-Gly-
Gln-Trp-Ser-Asn-Phe-Pro-Pro-Lys-Cys-Ile-Arg-Glu-Cys-Ala-Met-Val-Ser-Ser
SEQ ID NO: 3 (S3, Sushi-3) His-Ala-Glu-His-Lys-Val-Lys-Ile-Gly-Val-Glu-Gln-Lys-Tyr-
Gly-Gln-Phe-Pro-Gln-Gly-Thr-Glu-Val-Thr-Tyr-Thr-Cys-Ser-Gly-Asn-Tyr-Phe-Leu-Met
SEQ ID NO: 4 (SΔ3) His-Ala-Glu-His-Lys-Val-Lys-Ile-Lys-Val-Lys-Gln-Lys-Tyr-Gly-Gln-
Phe-Pro-Gln-Gly-Thr-Glu-Val-Thr-Tyr-Thr-Cys-Ser-Gly-Asn-Tyr-Phe-Leu-Met
SEQ ID NO: 5 (S4, Sushi-4) Arg-Ala-Glu-His-Lys-Val-Lys-Lys-Ile-Val-Lys-Gln-Leu-Tyr-
Gly-Gln-Phe-Arg-Gln-Leu-Thr-Arg-Val-Thr-Arg-Thr-Cys-Ser-Arg-Phe-Leu
SEQ ID NO: 6 (S5, Sushi-5) His-Lys-Val-Lys-Lys-Ile-Val-Lys-Gln-Leu-Tyr-Arg-Ala-Glu-
His-Lys-Val-Lys-Lys-Ile-Val-Lys-Gln-Leu
SEQ ID NO: 7 (Sushi-6-vg1) Met-Arg-Lys-Leu-Val-Leu-Ala-Leu-Ala-Lys-Ala-Leu-Ala-Lys-
Val-Asp-Lys-Lys-Asn-Leu
SEQ ID NO: 8 (Sushi-7-vg2) Leu-Leu-Asn-Ala-Val-Pro-His-Lys-Ala-Thr-His-Ala-Ala-Leu-
Lys-Phe-Leu-Lys-Glu-Lys
SEQ ID NO: 9 (Sushi-8-vg3) Gly-Val-Ser-Thr-Thr-Val-Leu-Asn-Ile-Tyr-Arg-Gly-Ile-Ile-
Asn-Leu-Leu-Gln-Leu-Asn-Val-Lys-Lys
SEQ ID NO: 10 (Sushi-8-vg3) Ile-Tyr-Arg-Gly-Ile-Ile-Asn-Leu-Ile-Gln-Leu-Ala-Val-Lys-
Lys-Ala-Gln-Asn-Val-Tyr-Gln-Met
SEQ ID NO: 11 (Segment 1) Boc-His(Trt)-Ala-Glu(OtBu)-His(Trt)-Lys(Boc)-Val-Lys(Boc)-
Ile-Gly-OH
SEQ ID NO: 12 (Segment 2) Fmoc-Val-Glu(OtBu)-Gln(Trt)-Lys(Boc)-Tyr(tBu)-Gly-Gln(Trt)-
Phe-Pro-Gln(Trt)-Gly-OH
SEQ ID NO: 13 (Segment 3) Fmoc-Thr(tBu)-Glu(OtBu)-Val-Thr(tBu)-Tyr(tBu)-Thr(tBu)-
Cys(Trt)-Ser(tBu)-Gly-OH
SEQ ID NO: 14 (Segment 4) Fmoc-Asn(Trt)-Tyr(tBu)-Phe-Leu-Met-OH
SEQ ID NO: 15 (Factor C) Carcinoscorpius rotundicauda
mwvtcfdtfl fvcessvfcl lcvwrfgfcr wrvfysfpfv kstvvllqcy hyslhntskf ysvnpdkpey ilsglvlgll
aqkmrpvqsk gvdlglcdet rfeckcgdpg yvfnipvkqc tyfyrwrpyc kpcddleakd icpkykrcqe
ckagldscvt cppnkygtwc sgecqckngg icdqrtgaca crdryegvhc eilkgcpllp sdsqvqevrn
ppdnpqtidy scspgfklkg marisclpng qwsnfppkci recamvsspe hgkvnalsgd miegatlrfs
cdspyyligq etltcqgngq wngqipqckn lvfcpdldpv nhaehkvkig veqkygqfpq gtevtytcsg
nyflmgfdtl kcnpdgswsg sqpscvkvad revdcdskav dflddvgepv rihcpagcsl tagtvwgtai
yhelssvcra aihagklpns ggavhvvnng pysdflgsdl ngikseelks larsfrfdyv ssstagksgc pdgwfevden
cvyvtskqra weraqgvctn maarlavldk dvipnsltet lrgkgltttw iglhrldaek pfiwelmdrs nvvlndnltf
wasgepgnet ncvymdiqdq lqsvwktksc fqpssfacmm dlsdrnkakc ddpgslengh atlhgqsidg
fyagssirys cevlhylsgt etvtcttngt wsapkprcik vitcqnppvp sygsveikpp srtnsisrvg spflrlprlp
lplaraakpp pkprssqpst vdlaskvklp eghyrvgsra iytcesryye llgsqgrrcd sngnwsgrpa scipvcgrsd
sprspfiwng nsteigqwpw qagisrwlad hnmwflqcgg sllnekwivt aahcvtysat aeiidpnqfk
mylgkyyrdd srdddyvqvr ealeihvnpn ydpgnlnfdi aliqlktpvt lttrvqpicl ptdittrehl kegtlavvtg
wglnenntys etiqqavlpv vaastceegy keadlpltvt enmfcagykk grydacsgds ggplvfadds rterrwvleg
ivswgspsgc gkanqyggft kvnvflswir qfi

What is claimed is:

1. A peptide-lipid conjugate comprising a peptide conjugated to a lipid, wherein the peptide comprises a sequence selected from Sushi-1 (SEQ ID NO:1), SΔ1 (SEQ ID NO:2), Sushi-3 (SEQ ID NO:3), SΔ3 (SEQ ID NO:4), Sushi-4 (SEQ ID NO:5), Sushi-5 SEQ ID NO:6), Sushi-6-vg1 (SEQ ID NO:7), Sushi-7-vg2 (SEQ ID NO:8), Sushi-8-vg3 (SEQ ID NO:9), Sushi-8-vg3 (SEQ ID NO:10), SEQ ID NO:11, SEQ ID NO:12, SEQ ID NO:13, and SEQ ID NO:14.

2. The peptide-lipid conjugate of claim 1, wherein the peptide comprises Sushi-3 (SEQ ID NO:3).

3. The peptide-lipid conjugate of claim 1, wherein the peptide-lipid conjugate is encapsulated in a polymer matrix.

4. The peptide-lipid conjugate of claim 1, wherein the peptide-lipid conjugate is encapsulated in an implant.

5. The peptide-lipid conjugate of claim 1, wherein the peptide-lipid conjugate is encapsulated in a microparticle.

6. The peptide-lipid conjugate of claim 5, wherein the microparticle comprises poly(lactide), poly(glycolide), or poly(lactide)-co-(glycolide).

7. The peptide-lipid conjugate of claim 1, wherein the peptide-lipid conjugate is encapsulated in a fiber.

8. The peptide-lipid conjugate of claim 1, wherein the peptide-lipid conjugate is encapsulated in a liposome.

9. The peptide-lipid conjugate of claim 1, wherein the peptide is conjugate to the lipid at the carboxy terminus of the peptide.

10. A pharmaceutical composition comprising the peptide-lipid conjugate of claim 1 and a pharmaceutically acceptable carrier.

11. The pharmaceutical composition of claim 10, wherein the pharmaceutically acceptable carrier comprises a microparticle.

12. A method for treating a disorder associated with lipopolysaccharide (LPS), comprising administering to a subject diagnosed with the disorder associated with LPS a peptide-lipid conjugate comprising a peptide that binds to LPS, in an effective amount to treat the disorder.

13. The method of claim 12, further comprising selecting a peptide that binds to LPS and conjugating the peptide with a lipid to form the peptide-lipid conjugate.

14. The method of claim 12, wherein the peptide comprises a sequence selected from Sushi-1 (SEQ ID NO:1), SΔ1 (SEQ ID NO:2), Sushi-3 (SEQ ID NO:3), SΔ3 (SEQ ID NO:4), Sushi-4 (SEQ ID NO: 5), Sushi-5 SEQ ID NO: 6), Sushi-6-vg1 (SEQ ID NO: 7), Sushi-7-vg2 (SEQ ID NO:8), Sushi-8-vg3 (SEQ ID NO:9), Sushi-8-vg3 (SEQ ID NO:10), SEQ ID NO:11, SEQ ID NO:12, SEQ ID NO:13, and SEQ ID NO:14.

15. The method of claim 12, wherein the peptide comprises Sushi-3 (SEQ ID NO:3).

16. The method of claim 12, wherein the peptide-lipid conjugate is encapsulated in a polymer matrix.

17. The method of claim 12, wherein the peptide-lipid conjugate is encapsulated in an implant.

18. The method of claim 12, wherein the peptide-lipid conjugate is encapsulated in a microparticle.

19. The method of claim 18, wherein the microparticle comprises poly(lactide), poly(glycolide), or poly(lactide)-co-(glycolide).

20. The method of claim 12, wherein the peptide-lipid conjugate is encapsulated in a fiber.

21. The method of claim 12, wherein the peptide-lipid conjugate is encapsulated in a liposome.

22. The method of claim 12, wherein the peptide is conjugated to the lipid at the carboxy terminus of the peptide.

23. The method of claim 12, wherein the disorder is a bacterial infection.

24. The method of claim 12, wherein the peptide-lipid conjugate is present in a pharmaceutical composition comprising a pharmaceutically acceptable carrier.

25. A method for delivering a conjugate, comprising administering to a subject a polymer matrix having encapsulated therein a peptide-lipid conjugate comprising a peptide that binds to lipopolysaccharide (LPS), under conditions effective to deliver the peptide-lipid conjugate to lipopolysaccharide in the subject.

26. The method of claim 25, further comprising selecting a peptide that binds to lipopolysaccharide (LPS) and conjugating the peptide with a lipid to form the peptide-lipid conjugate.

27. The method of claim 25, wherein the peptide comprises a sequence selected from Sushi-1 (SEQ ID NO:1), SΔ1 (SEQ ID NO:2), Sushi-3 (SEQ ID NO:3), SΔ3 (SEQ ID NO:4), Sushi-4 (SEQ ID NO: 5), Sushi-5 SEQ ID NO: 6), Sushi-6-vg1 (SEQ ID NO: 7), Sushi-7-vg2 (SEQ ID NO:8), Sushi-8-vg3 (SEQ ID NO:9), Sushi-8-vg3 (SEQ ID NO:10); SEQ ID NO:11, SEQ ID NO:12, SEQ ID NO:13, and SEQ ID NO:14.

28. The method of claim 25, wherein the peptide comprises Sushi-3 (SEQ ID NO:3).

29. The method of claim 25, wherein the polymer matrix is a polymer matrix of an implant.

30. The method of claim 25, wherein the polymer matrix is a polymer matrix of a microparticle.

31. The method of claim 30, wherein the microparticle comprises poly(lactide), poly(glycolide), or poly(lactide)-co-(glycolide).

32. The method of claim 25, wherein the polymer matrix is a polymer matrix of a fiber.

33. The method of claim 25, wherein the peptide is conjugated to the lipid at the carboxy terminus of the peptide.


SEQUENCE LISTING (14 sequences)

SEQ ID NO: 1 - protein, 34 aa, Artificial Sequence
  Description of Artificial Sequence Note = Synthetic Construct
GFKLKGMARISCLPNGQWSNFPPKCIRECAMVSS

SEQ ID NO: 2 - protein, 34 aa, Artificial Sequence
  Description of Artificial Sequence Note = Synthetic Construct
GFKLKGKAKISCLPNGQWSNFPPKCIRECAMVSS

SEQ ID NO: 3 - protein, 34 aa, Artificial Sequence
  Description of Artificial Sequence Note = Synthetic Construct
HAEHKVKIGVEQKYGQFPQGTEVTYTCSGNYFLM

SEQ ID NO: 4 - protein, 34 aa, Artificial Sequence
  Description of Artificial Sequence Note = Synthetic Construct
HAEHKVKIKVKQKYGQFPQGTEVTYTCSGNYFLM

SEQ ID NO: 5 - protein, 31 aa, Artificial Sequence
  Description of Artificial Sequence Note = Synthetic Construct
RAEHKVKKIVKQLYGQFRQLTRVTRTCSRFL

SEQ ID NO: 6 - protein, 24 aa, Artificial Sequence
  Description of Artificial Sequence Note = Synthetic Construct
HKVKKIVKQLYRAEHKVKKIVKQL

SEQ ID NO: 7 - protein, 20 aa, Artificial Sequence
  Description of Artificial Sequence Note = Synthetic Construct
MRKLVLALAKALAKVDKKNL

SEQ ID NO: 8 - protein, 20 aa, Artificial Sequence
  Description of Artificial Sequence Note = Synthetic Construct
LLNAVPHKATHAALKFLKEK

SEQ ID NO: 9 - protein, 23 aa, Artificial Sequence
  Description of Artificial Sequence Note = Synthetic Construct
GVSTTVLNIYRGIINLLQLNVKK

SEQ ID NO: 10 - protein, 22 aa, Artificial Sequence
  Description of Artificial Sequence Note = Synthetic Construct
IYRGIINLIQLAVKKAQNVYQM

SEQ ID NO: 11 - protein, 9 aa, Artificial Sequence
  Description of Artificial Sequence Note = Synthetic Construct
HAEHKVKIG

SEQ ID NO: 12 - protein, 11 aa, Artificial Sequence
  Description of Artificial Sequence Note = Synthetic Construct
VEQKYGQFPQG

SEQ ID NO: 13 - protein, 9 aa, Artificial Sequence
  Description of Artificial Sequence Note = Synthetic Construct
TEVTYTCSG

SEQ ID NO: 14 - protein, 5 aa, Artificial Sequence
  Description of Artificial Sequence Note = Synthetic Construct
NYFLM